(19) United States
(12) Patent Application Publication
(10) Pub. No.: US 2013/0025601 A1
(43) Pub. Date: Jan. 31, 2013

(54) TRACHEAL TUBE WITH REINFORCED PROXIMAL EXTENSION

(71) Applicants: Paul Waldron, Galway (IE); James Curley, Offaly (IE); James Stephenson, Galway (IE); Steven J. Grate, St. Peters, MO (US); Mark R. Behlmaier, Erie, CO (US); John Burns, Longmont, CO (US)

(72) Inventors: Paul Waldron, Galway (IE); James Curley, Offaly (IE); James Stephenson, Galway (IE); Steven J. Grate, St. Peters, MO (US); Mark R. Behlmaier, Erie, CO (US); John Burns, Longmont, CO (US)

(73) Assignee: Nelicor Puritan Bennett LLC, Boulder, CO (US)

(21) Appl. No.: 13/193,843

(22) Filed: Jul. 29, 2011

(51) Int. Cl. A61M 16/04

(52) U.S. Cl. 128/207.14

(57) ABSTRACT

A tracheal tube assembly includes a cannula configured to be positioned in a patient airway, the assembly further comprising a reinforcing member and/or extension member. The tracheal tube assembly further includes a flange member secured about the cannula. The tracheal tube assembly additionally includes a connector coupled to a proximal end of the cannula, wherein the cannula and the connector form a contiguous passageway for exchanging fluid with the patient airway in operation.

### BACKGROUND

The present disclosure relates generally to the field of tracheal tubes and, more particularly, to a tracheal tube having a proximal extension.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

Such tubes may include an inflatable balloon cuff, or may be cuffless. In both cases, a connector is typically provided at an upper or proximal end where the tube exits the patient airway. Standard connectors have been developed to allow the tube to then be coupled to artificial ventilation equipment to supply the desired air or gas mixture to the patient, and to evacuate gasses from the lungs.

One difficulty that arises in the use of tracheal tubes, and tracheostomy tubes in particular, is in the connection and manipulation of the proximal end of the tube. For example, endotracheal tubes may be fairly large in diameter, depending upon the size and age of the patient. Tracheostomy tubes, on the other hand, are typically kept fairly small to accommodate the routing of the tube through the passageway formed in the neck and trachea of the patient. This small size may lead to issues when a connection is made to ventilation equipment or when the tubes must be manipulated or held in place while a connection is fitted over a standard mating connector end formed on the tube. The orientation of the mating connector on the tube may make such connections difficult, and may cause movement of the tube in the patient during normal patient activity, even if somewhat constrained, and during movement of the patient, such as for changing of bed linens, surgical and imaging procedures, and so forth. In conventional tubes, the connector is provided relatively close to the neck of the patient, making connections somewhat more difficult.

There is a need, therefore, for improved tracheal tubes, and particularly for improved tracheostomy tubes. It would be desirable to provide a tube that allows for greater facility in making and changing connections with ventilation equipment while reducing the potential for bending or kinking of the tube at a proximal end.

### BRIEF DESCRIPTION

This disclosure provides a novel tracheal tube designed to respond to such needs. The tube allows for extension of a proximal end beyond a point where it exits the patient. In a tracheostomy tube embodiment, for example, a flange member fits adjacent to the neck of a patient and an extension is provided between this member and a standard connector. The extension and/or the tube may be reinforced to allow for manipulation of the tube and the connector while reducing the potential for bending or kinking. In certain embodiments, the extension may be removable, enabling the use of the extensions of various sizes. Additionally, the extension may be magnetic resonance imaging (MRI) compatible, thus allowing for artificial ventilation during MRI procedures.

Thus, in accordance with a first aspect, a tracheal tube assembly comprises a cannula configured to be positioned in a patient airway, the cannula comprising a reinforcing member. The tracheal tube assembly further comprises a flange member secured about the cannula. The tracheal tube assembly additionally comprises a connector coupled to a proximal end of the cannula, wherein the cannula and the connector form a contiguous passageway for exchanging fluid with the patient airway in operation.

In accordance with another aspect, a tracheal tube assembly comprises a cannula configured to be positioned in a patient airway and a flange member configured to be secured about the cannula. The tracheal tube assembly further comprises an extension tube having a reinforcing member, a first connector, and a second connector. The tracheal tube assembly additionally comprises an end connector configured to couple with the first connector of the extension tube, and a spigot configured to couple with the flange member comprising a proximal protrusion and a distal protrusion, wherein the proximal protrusion is configured to couple with the second connector of the extension tube and the distal protrusion is configured to couple with the cannula.

Also disclosed herein is a tracheal tube assembly comprising a first cannula configured to be positioned in a patient airway and flange member secured about the first cannula. The tracheal tube assembly further comprises a connector and a second cannula secured to the flange member comprising a reinforcing member, wherein a proximal portion of the second cannula comprises a proximal extension having the reinforcing member.

### BRIEF DESCRIPTION OF THE DRAWINGS

Various aspects of the disclosed techniques may become apparent upon reading the following detailed description and upon reference to the drawings in which:

FIG. 1 is a perspective view of an exemplary tracheal tube in accordance with aspects of the present techniques;

FIG. 1a is a side view of an embodiment of a connector of FIG. 1 having a fillet portion on a distal end taken within arc la-la;

FIG. 2 is a perspective view of certain functional parts of the tube shown in FIG. 1 prior to final assembly;

FIG. 3 is a sectional view of the final product shown in FIG. 1 illustrating a proximal extension and a reinforcing element within a later-formed connector;

FIG. 4 is sectional view of a tracheal tube having a reinforced cannula and a non-reinforced cannula;

FIG. 5 is an exploded sectional view of an embodiment of a tracheal tube assembly having a reinforced extension tube; and

FIG. 6 is a sectional view of the final product shown in FIG. 5.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

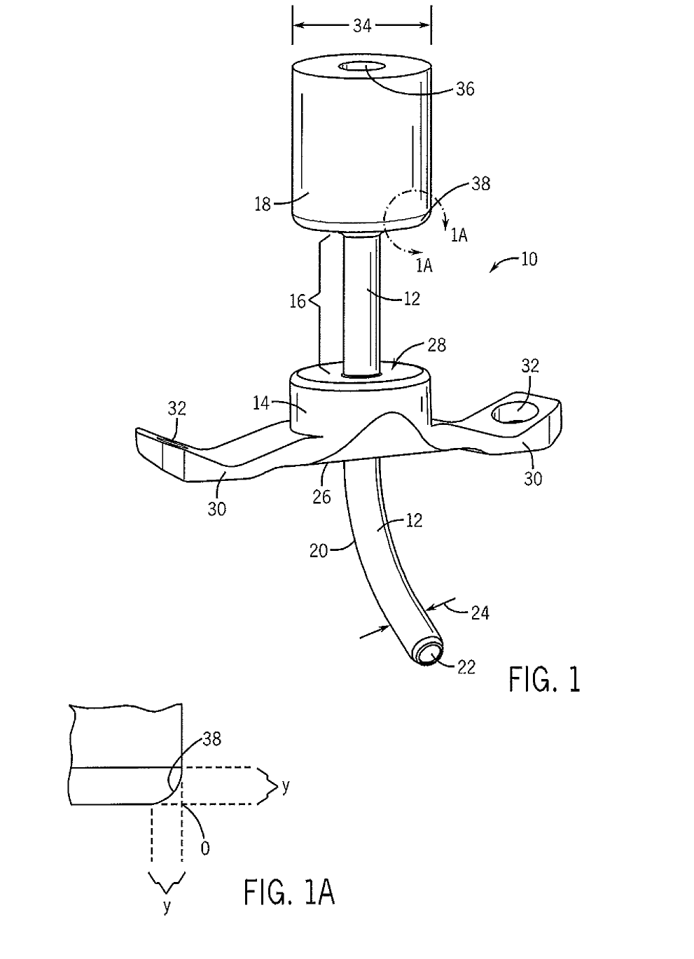

A tracheal tube according to a preferred embodiment is illustrated in FIG. 1. The tracheal tube assembly 10 represented in the figures is a tracheostomy tube, although aspects of this disclosure could be applied to other tracheal tube structures, such as endotracheal tubes. The application to a tracheostomy tube is apt, however, insomuch as such tubes tend to be smaller and more prone to bending, particularly if extensions are provided between the base of the tube and a connection as described below.

The assembly 10 includes a cannula 12 extending both distally as well as proximally from a flange member 14. A proximal portion 16 of the cannula 12 terminates in a connector 18, thus providing for an extension of the connector 18 from the flange member 14. In use, the cannula 12 is placed through an opening formed in the neck and trachea of a patient, and extends into the patient airway. The embodiment illustrated in the figures is free of outer seals or cuffs, although in practice a wide range of tube designs may be used, including tubes having one or more sealing cuffs around the cannula 12. Moreover, the cannula may include a single tube or nested tubes (e.g., disposable or reusable inner cannula and outer cannula), depending upon the assembly design. The cannula 12 in the illustrated embodiment forms a tube 20 through which a passageway 22 is provided. The cannula has an outer dimension 24 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, outer dimension 24 of the tube 20 may range from 4mm to 16mm.

In one embodiment, the cannula 12 enters the flange member 14 along a lower face 26 and protrudes through a passageway 28 of the flange member 14. When in use, the face 26 will generally be positioned against the neck of a patient, with the cannula extending through an opening formed in the neck and trachea. A pair of side flanges 30 extend laterally and serve to allow a strap or retaining member (not shown) to hold the tube assembly in place on the patient. In the illustrated embodiment, apertures 32 are formed in each side flange to allow the passage of such a retaining device. In many applications, the flange member 14 may be taped or sutured in place as well.

The proximal portion or extension 16 of the cannula 12 extends from the flange member 14 and allows for ease of access to the connector 18. The extension 16 essentially forms a neck or tubular section between the upper surface of the flange member and the lower surface of the connector. In presently contemplated embodiments, the extension between these surfaces may have a length ranging from approximately 15 mm to approximately 60 mm, although other lengths may be envisaged. Similarly, in the illustrated embodiments the proximal extension 16 is generally straight and cylindrical. In other configurations, however, the extension could be formed with bends, radiuses, and so forth.

The connector 18 is formed in accordance with industry standards to permit and facilitate connection to ventilating equipment (not shown). By way of example, standard outer dimensions may be provided as indicated at reference numeral 34 that allow a mating connector piece to be secured on the connector shown. By way of example, a presently contemplated standard dimension 34 accommodates a 15mm connector, although other sizes and connector styles may be used. An aperture 36 is formed in the connector 18 and is contiguous with cannula 12 and the passageway 22 formed in the cannula 12. In use, then, air or other gas may be supplied through the connector, the proximal extension and the cannula, and gasses may be extracted from the patient. For enhanced patient comfort, the connector 18 may include a fillet portion 38, as described in more detail below with respect to FIG. 1a. In other embodiments, a non-fillet portion may be included.

FIG. 1a is a side view illustrating a generally curved edge or fillet portion 38 disposed on the distal end of the connector 18. Advantageously, the fillet portion 38 may prevent irritation associated with a contact or rubbing of the distal end of the connector 18 against the patient's anatomy, such as the chin or neck. By providing for a smooth, fillet edge 38 disposed circumferentially about the connector's distal end, contact with the patient's chin or neck may be minimized or eliminated. In the depicted embodiment, the fillet portion 38 may be approximately defined by using one of the convex sides (e.g., second quadrant side) of an astroid shape with an origin o and having the parametric equation x=cos³ θ, y=sin³ θ, for 0≦0≦2Π. Other convex-defining equations may be used to define the fillet portion 38, including circular curves, ellipsoid curves, and more generally, equations defining sloping surfaces.

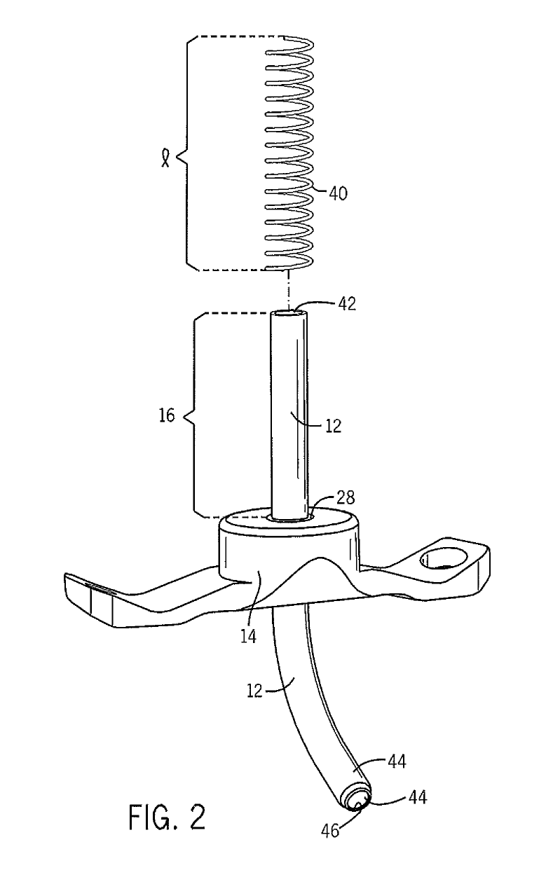

FIG. 2 illustrates elements of the tracheal tube assembly 10. In the illustrated embodiment, the cannula 12 includes a reinforcing member 40. The reinforcing member 40 may have a length l equal to a length of the cannula 12 as measured from a proximal opening 42 to a distal opening 44 of the cannula 12. That is, in the presently contemplated embodiment, the reinforcing member 40 may reinforce the entire length of the cannula 12. In another embodiment, the reinforcing member 40 may reinforce only a portion of the cannula 12. For example, the reinforcing member 40 may reinforce approximately 10%-20%, 10%-40%, 5%-90% of the cannula 12.

The reinforcing member 40 may provide reinforcement against bending, kinking, and deformation. In the illustrated embodiment, the reinforcing member 40 is a helical or coil-like piece that is disposed within the cannula 12. That is, the reinforcing member 40 may be disposed between outer walls 44 and inner walls 46 of the cannula 12. In certain embodiments, the reinforcing member may comprise, for example, longitudinal strips or strands or material within or adjacent to the cannula 12 that resist substantial bending and kinking. Moreover, various types of matrices (e.g., crisscrossed beads or strands) may be used around and within the cannula 12 to serve as the reinforcing member. In one embodiment, the cannula 12 is made of polyvinylchloride, and the reinforcing member 40 is made of a metal or metal alloy, such as stainless steel. The materials used for these components may vary, however, and acceptable materials for cannulae may include, by way of example, a PEBAX silicone, or a polyurethane. The materials for reinforcing member 40 may also include Nitinol or chromoly. In certain embodiments, it may be preferred that the reinforcing member 40 be made of a non-ferromagnetic material such that the entire tube assembly may be left in place during certain imaging procedures, such as magnetic resonance imaging (MRI) procedures. Accordingly, the reinforcing member 40 may be made of a nylon or a phosphor bronze.

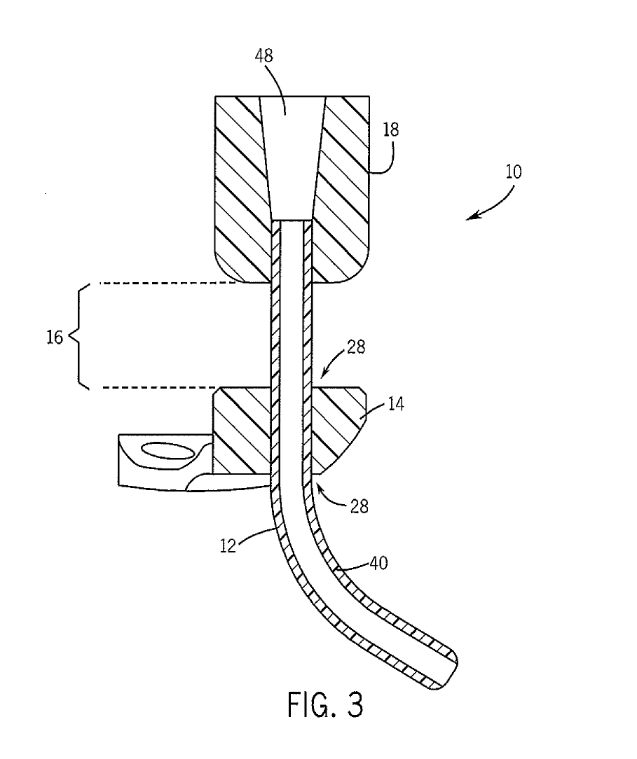

FIG. 3 sectional side view illustrating a final stage in the manufacture of the tracheal tube assembly 10 in accordance with one embodiment. While the flange member 14 may be an assembly of components, in the illustrated embodiment it is a single molded piece, such that the passageway 28 can be formed by the die in which the flange member 14 is molded. The cannula 12 having the reinforcing member 40 may then be installed through the passageway 28 in the flange member 40. In one embodiment, the cannula 12 may be bonded to the flange member 40 by using, for example, thermal bonding, adhesives, and the like,

In other embodiments, the cannula 12 may be re-positionable with respect to the flange member 14. That is, the cannula 12 may be moved by a clinician or user in order to increase or decrease the length of the proximal extension 16. In these embodiments, the cannula 12 may be secured by non-bonding techniques. For example, an interference fit between the cannula 12 and the passageway 28 may securely bind the cannula 12 to the flange member 14 while enabling the clinician or user to reposition the cannula 12 with respect to the flange member 14, thus adjusting the length of the proximal extension 16. Likewise, threads or grooves may be molded or overmolded in the passageway 28, with corresponding threads or grooves in the exterior surfaces of the cannula 12, to allow for threading or unthreading of the cannula 12 through the passageway 28. In this manner, the proximal extension 16 may be adjusted to desired lengths.

In the illustrated embodiment, the connector 18 includes a tapered passageway 48 formed by the die in which the connector 18 is molded. Thereafter, the connector 18 may be installed. In one embodiment, the cannula 12 may be pressed inside the tapered passageway 48 and secured by an interference fit. In other embodiments, the cannula 12 may be bonded or otherwise adhered to the passageway 48. Other embodiments may call for inserts, collars, transition elements, and so forth in the connector 18 that may be inserted into a mold during the same process, or that may be inserted, assembled or affixed in separate operations. In a presently contemplated embodiment, the connector 18 is formed of polyvinylchloride or PEBAX, although other suitable materials may be employed. Once completed, the structure may appear as illustrated.

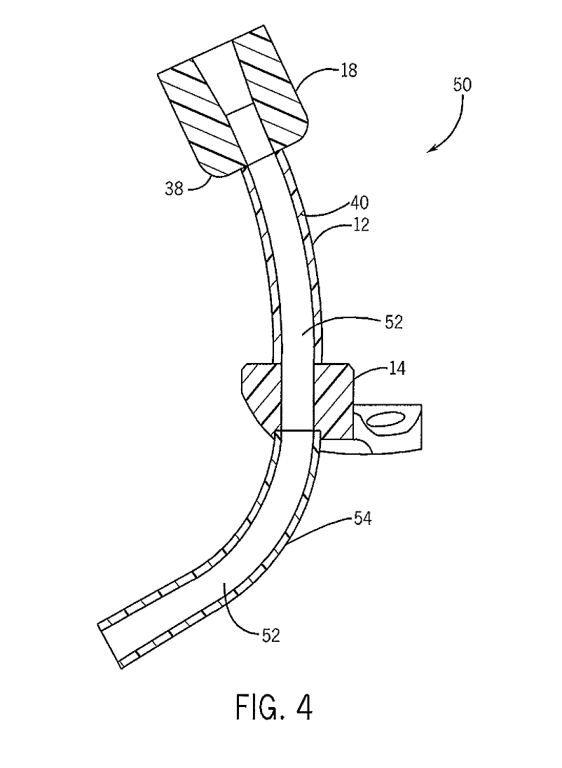

FIG. 4 is a sectional side view of an embodiment of a tracheal tube assembly 50 in which a tube 52 is formed by coupling the reinforced cannula 12 with a non-reinforced cannula 54. In the presently contemplated embodiment, the assembly 50 includes the reinforced cannula 12 extending proximally from the flange member 14, with the non-reinforced cannula 54 extending distally. The cannulae 12 and 54 may then be bonded or otherwise attached to the flange member 14, forming the continuous passageway tube 52.

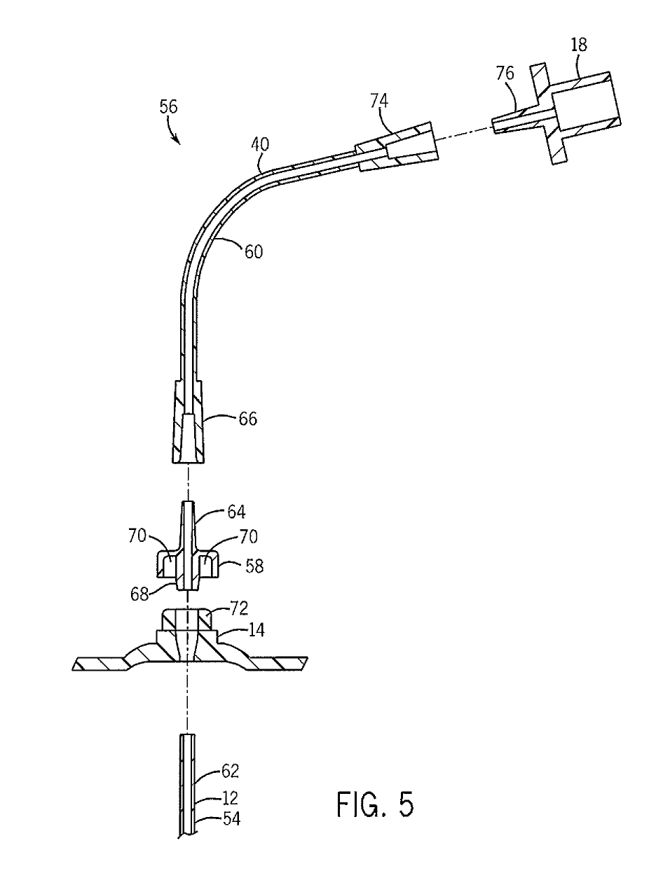

The non-reinforced cannula 54 may be extruded, molded, or overmolded out of a polyvinylchloride, a PEBAX silicone, a polyurethane, or other suitable material, and may then be bonded to the distal end of the flange member 14. The reinforced cannula 12 may be manufactured as described above with respect to FIGS. 1-3. That is, the reinforced member 40 is disposed within the reinforced cannula 12 to provide reinforcement against bending, kinking, and deformation. Likewise, the flange member 14 may be manufactured as described above, to provide for a platform from which to attach the cannulae 12 and 54. The end connector 18 may be bonded or otherwise adhered to a proximal end of the reinforced cannula 12, and may also include the fillet portion 38, as shown. In other embodiments, a spigot may be used to couple a proximal extension to a distal cannula, as described in more detail with respect to FIG. 5 below.

FIG. 5 is an exploded section view of an embodiment of a tracheal tube assembly 56 in which a spigot 58 is used to couple a reinforced extension tube 60 to a cannula 62 (e.g., non-reinforced cannula 54 or reinforced cannula 12). In one embodiment, the spigot 58 enables the manufacturing reuse of the flange member 14 by using the flange member 14 as a base platform to connect the extension tube 60 with the cannula 62. The spigot 58 includes a proximal protrusion or nipple 64 suitable for coupling with a female connector 66 of the extension tube 60. Likewise, a distal protrusion or nipple 68 is provided, suitable for coupling with the cannula 62. A chamber 70 is also provided, that mates with a corresponding protrusion 72 of the flange member 14. By enabling the coupling of the extension tube 60 with the flange member 14, the spigot 58 provides for the reuse of the flange member 14 as a coupling flange base for the extension tube 60 and the cannula 62.

The spigot 58 may be may be extruded, molded, or overmolded out of a polyvinylchloride, a PEBAX silicone, a polyurethane, or other suitable material. In one embodiment, the spigot 58 may be bonded or adhered to the flange member 14. In another embodiment, the spigot 58 may be coupled to the flange member 14 by using an interference fit (e.g., press or friction tit). In the presently contemplated embodiment, the extension tube 60 includes the reinforcing member 40. In other embodiments, the extension tube 60 is not reinforced. The extension tube 60 may also be extruded, molded, or overmolded out of, for example, a polyvinylchloride, a PEBAX silicone, or a polyurethane. Further, the extension tube 60 may have a length ranging from approximately 15 mm to approximately 60 mm, with an internal diameter (ID) between 1 mm to 10 mm, although other lengths and internal diameters may be envisaged.

In certain embodiments, the spigot 58 may be provided as part of a tracheal tube kit. Indeed, some or all of the depicted assembly components 14, 18, 58, 60, and 62, may be provided in a kit for final assembly at a patient care site. The components 14, 18, 58, 60, and 62 may then be affixed at the patient care site to custom fit certain patient anatomies. For example, tubes 60 of differing lengths and internal diameters may be selected based on the patient's physiology. Similarly, and ID of the tube 60 may be selected to maximize gas transmission and minimize work of breathing (WOB). During assembly by the clinician or user, the flange member's protrusion 72 may be manually inserted into the spigot's chamber 70, thus securing the spigot 58 to the flange member 14 via interference fit. Likewise, the spigot's proximal protrusion 64 may be inserted into the tube's female connector 66, The connector 18 (e.g., 15 mm end connector) may then be inserted into the tube's female connector 74 via the connector's distal protrusion 76. It is to be noted that, in one embodiment, the female connector 66 is identical to the female connector 74. In other embodiments, the female connector 66 and 74 may include different shapes and sizes. Additionally, the components 40, 60, 66, and 74 may be manufactured as a single component, for example, by molding or overmolding. In the depicted embodiment, the connector 18 does not include the fillet portion 38 shown in FIG. 1. However, in other embodiments, the fillet portion 38 may be included.

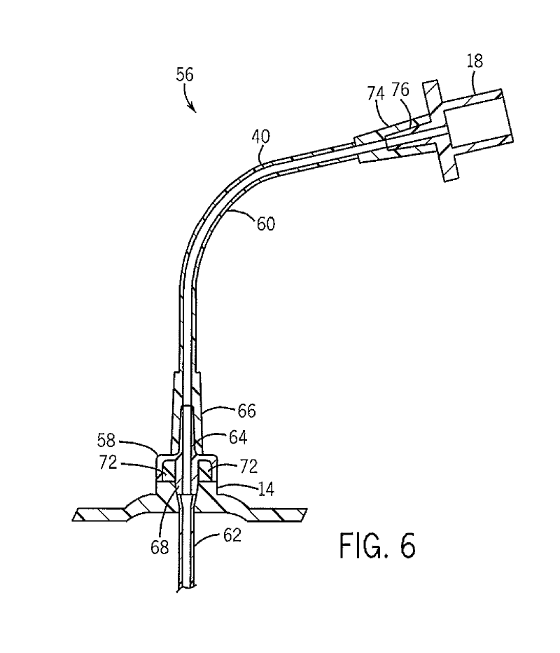

The protrusion 64, 68, and/or 76 may include locking features such as projections, barbs, and the like, with complementary grooves or notches included in the female connector 66, cannula 62, and/or female connector 74, to improve attachment. In certain embodiments, the cannula 62 may be pre-attached to the flange member 14 at the factory, for example, by bonding or adhering the cannula 62 to the flange member 14. Once completed, the structure may appear as illustrated in FIG. 6.

FIG. 6 is a sectional view of the finished tracheal tube assembly 56 shown in FIG. 5. It should be noted that in one embodiment, the tube assembly 56 may have been pre-assembled in its entirety in the factory, and is delivered as depicted in FIG. 6. In this embodiment, the components 14, 18, 58, 60, and 62 may be assembled through a variety of techniques, including thermal bonding, adhesives, interference fit, or a combination thereof, to provide for the fully assembled tube assembly 56. In other kit embodiments, the tracheal tube assembly 56 may be delivered partially assembled as a kit, which may then be fully assembled at the clinical site. In one kit example, the flange member 14, spigot 58, and cannula 62 may be delivered pre-assembled from the factory. The clinician may then select the tube 60 and connector 18 based on, for example, the patient's age, neck anatomy, and desired ventilator usage, and complete the assembly of the tube assembly 56. By providing for various types of tracheal tube assembly kits, the components described herein enable a more flexible, custom fitting of tracheal tubes to a wider diversity of patient anatomies.

In certain embodiments, the extension tube 60 may be detached, for example, during MRI procedures. In one embodiment, the spigot 58 and extension tube 60 may be detached from the flange member 14 before the MRI procedure, and subsequently reattached after termination of the procedure. In this embodiment, the spigot 58 may be affixed to the flange member 14 by an interference fit. The clinician may manually pull on the spigot 58 outwardly from the flange member 14, thus removing the spigot 58, extension tube 60, and connector 18. In another embodiment, the tube 60 may be similarly removed, but the spigot 58 left affixed to the flange member 14. In yet another embodiment, the reinforcing member 40 is manufactured out of an MRI-compatible material (e.g., nylon, phosphor bronze) and the extension tube 60 is not disconnected during the MRI procedure.

What is claimed is:

1. A tracheal tube assembly comprising:
a cannula configured to be positioned in a patient airway, comprising a reinforcing member;
a flange member secured about the cannula; and
a connector coupled to a proximal end of the cannula, wherein the cannula and the connector form a contiguous passageway for exchanging fluid with the patient airway in operation.

2. The assembly of claim 1, wherein a proximal portion of the cannula comprises a proximal extension having a length of 15mm to 60mm between a distal end of the connector and a proximal end of the flange member.

3. The assembly of claim 2, wherein the cannula is configured to be reposition able with respect to the flange member to vary the length of the proximal extension.

4. The assembly of claim 1, wherein the reinforcing member comprises a length equal to a full length of the cannula.

5. The assembly of claim 1, wherein the reinforcing member comprises a length 10% to 20%, 10% to 40%, or 5% to 90% of a full length of the cannula.

6. The assembly of claim 1, wherein the reinforcing member comprises a helical element.

7. The assembly of claim 6, wherein the helical element comprises a synthetic plastic material.

8. The assembly of claim 7, wherein the synthetic plastic material comprises nylon.

9. The assembly of claim 6, wherein the helical element comprises a metal.

10. The assembly of claim 9, wherein the metal comprises a phosphor bronze, a stainless steel, a chromoly, a Nitinol, or a combination thereof.

11. The assembly of claim 1, wherein the connector comprises a sloping surface circumferentially disposed on a distal end of the connector.

12. The assembly of claim 11, wherein the sloping surface comprises an astroid shape.

13. A tracheal tube assembly comprising:
a cannula configured to be positioned in a patient airway;
a flange member configured to be secured about the cannula;
an extension tube comprising a first connector, and a second connector;
an end connector configured to couple with the first connector of the extension tube; and
a spigot configured comprising a proximal protrusion and a distal protrusion, wherein the proximal protrusion is configured to couple with the second connector of the extension tube and the distal protrusion is configured to couple with the cannula or flange member.

14. The assembly of claim 13, wherein the assembly of claim 13 is provided as a tracheal tube kit configured to be assembled at a clinical site.

15. The assembly of claim 14, wherein the cannula is provided pre-assembled to the flange member as a first component of the tracheal tube kit.

16. The assembly of claim 15, wherein the end connector is provided pre-assembled to the extension tube as a second component of the tracheal tube kit.

17. The assembly of claim 14, wherein the spigot is provided pre-assembled to the flange member and to cannula as a first component of the tracheal tube kit.

18. The assembly of claim 15, wherein the extension tube comprises a reinforcing member.

19. A tracheal tube assembly comprising:
a first cannula configured to be positioned in a patient airway;
a flange member secured about the first cannula;
a connector; and
a second cannula secured to the flange member comprising a reinforcing member, wherein a proximal portion of the second cannula comprises a proximal extension having the reinforcing member.

20. The assembly of claim 19, wherein the reinforcing member comprises a helical element.
